SEQUENCE LISTING (17 sequences)

SEQ ID NO: 1 - DNA, 28 bp, Human Poxvirus
gcttccagattatgtgatagcaagacta

SEQ ID NO: 2 - DNA, 24 bp, Human Poxvirus
tccggatactccgtatcctattcc

SEQ ID NO: 3 - DNA, 16 bp, CrmB encoding viral analog of the cellular receptor for tumor necrossis factor
ctaayacaaacacaca

SEQ ID NO: 4 - DNA, 16 bp, ARTIFICIAL
  Oligonucleotide for use in differential diagnosis of orthopoxviruses
ctaayacaaaatgtac

SEQ ID NO: 5 - DNA, 16 bp, ARTIFICIAL
  Oligonucleotide for use in differential diagnosis of orthopoxviruses
ctaayacacgatgtac

SEQ ID NO: 6 - DNA, 13 bp, ARTIFICIAL
  Oligonucleotide for use in differential diagnosis of orthopoxviruses
ttacccgcttgtc

SEQ ID NO: 7 - DNA, 14 bp, ARTIFICIAL
  Oligonucleotide for use in differential diagnosis of orthopoxviruses
ttacaggcttgtct

SEQ ID NO: 8 - DNA, 14 bp, ARTIFICIAL
  Oligonucleotide for use in differential diagnosis of orthopoxviruses
ttacccacttgtct

SEQ ID NO: 9 - DNA, 16 bp, ARTIFICIAL
  Oligonucleotide for use in differential diagnosis of orthopoxviruses
aagatgcaatagtaat

SEQ ID NO: 10 - DNA, 16 bp, ARTIFICIAL
  Oligonucleotide for use in differential diagnosis of orthopoxviruses
aagatgcgatagtaat

SEQ ID NO: 11 - DNA, 16 bp, ARTIFICIAL
  Oligonucleotide for use in differential diagnosis of orthopoxviruses
aagatgtgatagtaat

SEQ ID NO: 12 - DNA, 12 bp, ARTIFICIAL
  Oligonucleotide for use in differential diagnosis of orthopoxviruses
ggaagacgcgat

SEQ ID NO: 13 - DNA, 14 bp, ARTIFICIAL
  Oligonucleotide for use in differential diagnosis of orthopoxviruses
gtcttcttaaagga

SEQ ID NO: 14 - DNA, 14 bp, ARTIFICIAL
  Oligonucleotide for use in differential diagnosis of orthopoxviruses
gtattctcaaagga

SEQ ID NO: 15 - DNA, 14 bp, ARTIFICIAL
  Oligonucleotide for use in differential diagnosis of orthopoxviruses
gtcttctcaaagga

SEQ ID NO: 16 - DNA, 14 bp, ARTIFICIAL
  Oligonucleotide for use in differential diagnosis of orthopoxviruses
atttcccaaacaaa

SEQ ID NO: 17 - DNA, 14 bp, ARTIFICIAL
  Oligonucleotide for use in differential diagnosis of orthopoxviruses
atttctaaaacaaa

(19) United States
(12) Patent Application Publication
(10) Pub. No.: US 2006/0147905 A1
(43) Pub. Date: Jul. 6, 2006

(54) Method for the specific identification of orthopoxvirus with the aid of a miniature biological chip

(76) Inventors: Andrei Darievich Mirzabekov, Moscow (RU); Natalia Vladimirovna Mirzabekova, Moscow (RU); Lev Stepanovich Sandakhchiev, Koltsovo (RU); Vladimir Mikhailovich Mikhailovich, Moscow (RU); Sergei Anatolievich Lapa, Konakovo (RU); Maxim Vyacheslavovich Mikheev, Koltsovo (RU); Sergei Nikolaevich Schelkunov, Koltsovo (RU)

Correspondence Address: LADAS & PARRY, 26 WEST 61ST STREET, NEW YORK, NY, 10023, US

(21) Appl. No.: 10/495,882

(22) PCT Filed: Nov. 26, 2001
(86) PCT No.: PCT/RU01/00507

§ 371 Date: May 10, 2005

(51) Int. Cl. C12Q 1/70; C12Q 1/68; C12M 1/34

(52) U.S. Cl. 435/5; 435/6; 435/287.2

(57) ABSTRACT

The invention relates to an alternative express-method for specific identification of orthopoxviruses with the aid of microchips by hybridising DNA-DNA. Said method involves a two-stage PCR producing a single stained fluorescently labelled DNA fragment, a hybridisation on a biochip containing an original set of typing oligonucleotides and original procedures for recording and interpreting results.

### FIELD OF THE INVENTION

The invention relates to the fields of molecular biology and virology, more specifically, to the identification of virus species of the Orthopoxvirus genus comprising the step of biochip hybridization of an appropriately treated sample of viral DNA. The invention also discloses the construction of highly specific DNA probes able to differentiate orthopoxvirus species according to the sequence of the crmB gene.

### BACKGROUND OF THE INVENTION

The following methods are currently available for the identification of orthopoxvirus species:
I. Detection of a poxvirus antigen by immunofluorescence [1, 2]
II. Electron immunomicroscopy [3, 4]
III. Propagation in chick embryos [5, 6]
IV. Enzyme-linked immunoassay as related to identification of orthopoxvirus species [2, 7]
V. Indirect hemagglutination assay [8]
VI. PCR-based methods:

a) with subsequent length analysis of the amplified fragment [9]

b) with subsequent restriction analysis [9, 10].

The efficiency of immunofluorescence methods is dependent on the type of test material. The results of analysis of the material from pustules and scabs is of low significance due to the autofluorescence of leukocytes present in the sample.

Methods II and IV require species-specific monoclonal antibodies for each orthopoxvirus species. Currently, the use of these methods has been validated for only a limited number of orthopoxviruses. Furthermore, the use of methods I, II and IV for species diagnostics is limited in view of the close antigenic relatedness between the various orthopoxviruses.

Propagation in chick embryos (III), being a culture method per se, requires specialized equipment and highly qualified staff. The analysis takes several days to perform and represents an inherent biological hazard. Furthermore, the method is sensitive to the quality and age of the chick embryos used and the results are often difficult to interpret.

Method V not only requires monoclonal antibodies, but also uses appropriately treated sheep red blood cells. The method has all the drawbacks inherent to methods I, II and IV, moreover, its sensitivity is rather low.

The drawbacks mentioned above are obviated by the present invention.

### SUMMARY OF THE INVENTION

The present invention provides an alternative, rapid method of species identification for six species (and two subspecies) of the Orthopoxvirus genus. As the target for differential diagnostics, the gene crmB encoding a viral analog of the cellular receptor for tumor necrosis factor was chosen. The method is based on a two-step PCR to obtain a single-stranded, fluorescently labeled DNA fragment with a subsequent hybridization step using a biochip that contains a set of differentiating oligonucleotides; it also includes procedures for detection and data processing.

The method is implemented by using a biological microchip produced as a microarray comprising a glass support with gel spots. Each gel spot contains a single typing oligonucleotide.

The sample to be hybridized with oligos immobilized on the chip is prepared by obtaining a single-stranded, fluorescently labeled PCR product by a two-step PCR reaction. The first PCR step is performed to amplify the crmB gene fragment of interest, the second step referred to as asymmetrical PCR is directed at preferential amplification of one DNA strand by using the amplicon from the first PCR step and a primer labeled with a fluorescent dye, such as Texas Red®.

The hybridization of sample DNA with oligos of the microchip is carried out by incubating the hybridization mix in a sealed reaction vessel over the chip for several hours at a given temperature. The oligonucleotides immobilized on the microchip are characterized by species specificity to various Orthopoxvirus species.

The detection of fluorescent signals is accomplished from the dried and washed-out microchip by using a detector such as, e.g., an experimental device comprising a fluorescence microscope, a CCD (charge-coupled device) camera, and a computer, or a portable device employing an exciting laser beam wherein the detection of fluorescent signals is accomplished by using a photographic film. The analysis of the hybridization pattern is performed by comparison to a set of standardized patterns specific for each Orthopoxvirus species.

The main advantages of the claimed method for orthopoxvirus identification by hybridizing a sample of amplified, fluorescently labeled DNA to a microchip are as follows:
high reliability based on the concurrent analysis of several species-specific regions of the viral crmB gene
rapid assay procedure
simple technique that obviates the need to employ highly qualified staff
relative cost-effectiveness.

Another object of the present invention is a test kit for the claimed method comprising:

(a) a microchip that contains the typing oligonucleotides and is used for the diagnostics of orthopoxvirus species, and

(b) optionally, reagents for DNA isolation, primers for PCR reactions (to amplify a specific fragment of the viral crmB gene), reagents for hybridization, and a device for the detection of assay results (such as a portable chip reader that employs an exciting laser beam and is equipped with a photographic camera or a CCD camera).

Yet another object of the present invention is a biological microchip used in the claimed method for identification of orthopoxvirus species, wherein said microchip is a microarray of gel spots on a glass support with immobilized typing oligonucleotides.

Still another object of the present invention comprises discriminating oligonucleotides intended to be immobilized on a biochip and characterized by species specificity (complementarity) to various orthopoxvirus species/

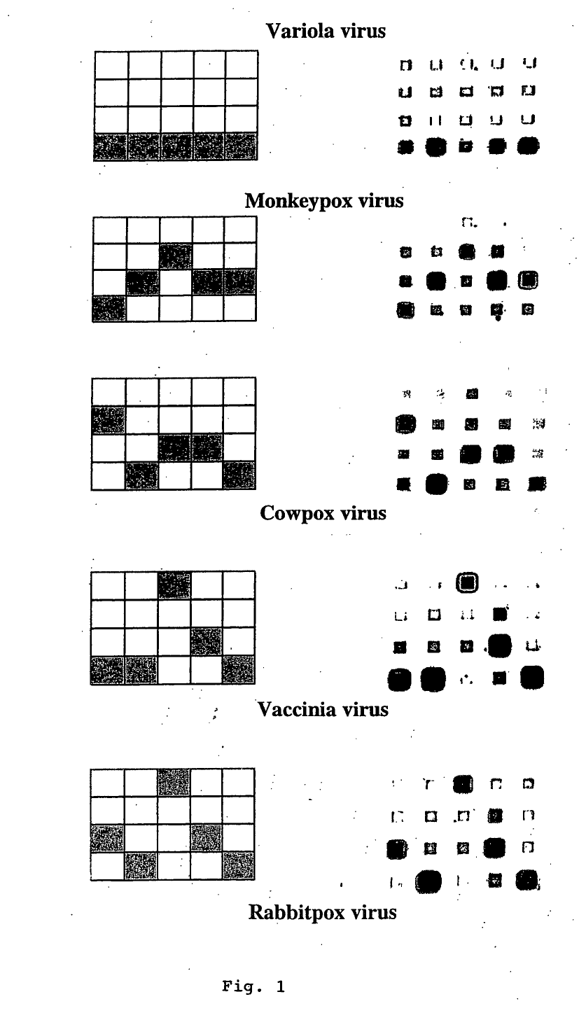

The invention is further explained in FIG. 1, where hybridization patterns are shown as compared with reference patterns.

The terms and definitions used in the description of the invention are intended to mean the following:

Biological microchip (biochip, DNA chip) is a support plate having an area of about 1 cm², on which, in certain order, are located gel spots that contain immobilized single-stranded oligonucleotides characterized by a unique sequence of nucleobases. The number of spots may vary up to 1 million per cm².

Antigen is a substance recognized as foreign by the living organism and able to elicit a specific immune response.

Antibody is a serum protein (immunoglobulin) found in the blood of humans and warm-blooded animals, which is able to bind an antigen specifically. By interacting with microorganisms, antibodies prevent them from proliferating or neutralize toxic substances produced by them.

PCR (polymerase chain reaction) is an enzymatic reaction utilizing the ability of DNA polymerases to synthesize a new DNA chain by using an existing DNA molecule as template on the basis of complementarity. The use of PCR makes it possible to increase up to several thousand times the amount of a test fragment in the sample, thus enhancing the sensitivity of PCR-based methods of diagnostics accordingly.

Amplification is used in molecular biology to denote the increase in the amount of DNA in the course of PCR.

### DETAILED DESCRIPTION OF THE INVENTION

The present invention is directed at providing a rapid method for identification of orthopoxvirus species on biochips. The method provides for typing at species-specific regions of the crmB gene, which is advantageous by allowing the identification of six species of said genus. Also provided is a set of species-specific oligonucleotide probes immobilized on a chip. The use of conventional methods for the isolation of viral DNA makes it possible to analyze natural samples, including human and animal tissues, for the presence of different viruses from the Orthopoxvirus genus. The method can be used for the diagnostics in the field conditions, if special equipment is available.

The flowsheet for the differential diagnostics of orthopoxviruses on a biochip comprises the following steps:

I. Design of a Biochip for the Identification of Orthopoxvirus Species

1) The Choice of Target

The product of crmB gene, a viral analog of the cellular receptor for tumor necrosis factor in humans and some mammals, is one of the main virulence factors for this group of viruses. When secreted by an infected cell, it binds the cellular tumor necrosis factor, thereby inhibiting the cytokine-mediated immune response. Species specificity of the above mammalian cytokine, as reflected in the structure of viral receptor analogs, determines a narrow host specificity for most orthopoxviruses. Therefore, the crmB gene contains conservative species-specific regions suitable to differentiate the species.

2. DNA Isolation

The DNA is isolated from natural samples (human and animal tissues) using the DNA isolation methods described for animal cells.

3. The Choice of Primers and Typing oligonucleotides

The primers are chosen so as to meet the requirements for high specificity to conserved regions of the orthopoxviral crmB gene, which makes it possible to selectively amplify the fragment of interest directly from natural samples, as well as to meet the requirements generally set for primers, i.e. the absence of stable secondary structures and the melting temperature differences not exceeding 3 to 4° C. The typing oligonucleotides are characterized by species specificity to different orthopoxvirus species.

II. Preparation of a Microarray for the Immobilization of Oligonucleotides to Produce a Biochip

A microarray of gel spots 100×100×20 μm in size was prepared by photopolymerizing a 5% polyacrylamide gel as previously described [11]. The gel was subjected to activation, then the oligonucleotides in solutions were applied and covalently linked to the active groups of the gel.

III. Sample Preparation for Analysis

A fragment of the crmB gene is amplified using highly specific primers. The amplified product is then used to produce fluorescently labeled, single-stranded DNA for a subsequent microchip hybridization. For this, the so-called method of asymmetrical PCR is used.

IV. Hybridization Procedure

The fluorescently labeled PCR product as described above is then hybridized with the differentiating oligos of the microchip in an appropriate buffer for 4 to 6 hours.

V. Detection and Analysis of Hybridization Results

The differentiating oligonucleotides are subdivided into five groups according to species-specific regions of the crmB gene. The intensity of fluorescent signals emitted by perfectly matched duplexes within a group is substantially higher than the intensity of signals from incompletely matched duplexes, which enables a reliable identification of orthopoxvirus species. The results are analyzed according to the hybridization pattern obtained by using an experimental device comprising, for example, a fluorescence microscope, a CCD camera, and a computer.

Experimental Procedures

Oligonucleotides

Oligonucleotides to be immobilized on a biochip were synthesized in an automated DNA/RNA synthesizer model 394 (Applied Biosystems) using a conventional phosphoamidite chemistry. 3′-Amino-Modifier C7 CPG 500 was used in the synthesis of oligonucleotides for a hybridization microchip so that the oligonucleotides synthesized contained a free amine group spacer at the 3′-end. The PCR primers were modified at the 5′-end by using 5′-Amino-Modifier C6 (or C12) (Glen Research, Va.).

Preparation of the Microarray for a Microchip

The microarray of gel spots 100×100×20 μm in size was prepared by photopolymerizing a 5% polyacrylamide gel as previously described [11].

The gel was activated with 2% trifluoroacetic acid at room temperature for 10 min, washed with water and dried. Thereafter the gel was -exposed sequentially to Repel-Silane (a 2% w/v solution of dimethyldichlorosilane in 1,1,1-trichloroethane, LKB Produkter AB, Bromma, Sweden) for 10 sec, dichloromethane for 10 sec and ethanol (95% v/v) for 10 sec, and then was washed with water for 3 min and dried.

Oligonucleotide solutions at 1 mM were spotted in duplicate using a robotic device in a volume of 1 nl. To stabilize the covalent bonds between the amine groups of spacers flanking one end of the oligos and aldehyde groups of the gel, the matrix was placed in a 0.1 M solution of pyridine-borane complex (Aldrich Chemical Co., Inc., Milwaukee, Wis.) in chloroform, overlaid with a water phase and allowed to stand for 12 to 16 h at room temperature. Then the biochip was rinsed with ethanol (95% v/v) and water. The unreacted aldehyde groups were reduced by treatment with a freshly made 0.1 M solution of NaBH₄ (Aldrich) for 20 min at room temperature. The biochip was rinsed with water, dried and stored at room temperature.

Preparation of Materials to be Amplified

The DNAs were isolated from natural sources (human and animal tissues) by using isolation methods described for DNA isolation from animal cells.

Amplification of crmB Gene Fragments and Production of Single-Stranded Labeled PCR Products

The preparation of viral DNA for hybridization was carried out by a two-step PCR in a GeneAmp PCR system 2400 (Perkin Elmer, Foster City, Calif., USA). The primers used for amplification were TNFR1f and TNFR3r flanking a fragment of 267 bp for human poxvirus, but varying in length for other species.

The first PCR step is carried out to produce the chosen fragment of viral DNA. The reaction buffer contained 16.6 mM (NH₄)₂SO₄, 67 mM Tris-HCl, pH 8.6 at 25° C., 1.75 mM MgCl₂, 120 μM dNTPs, 0.1% Triton X-100, 1.5 units of Taq DNA polymerase (Sileks, Moscow, Russia) and 5 pmoles each of the primers TNFR1f and TNFR3r in a volume of 30 μl.

The primers:

TNFR1f
5′-GCT TCC AGA TTA TGT GAT AGC AAG ACT A-3′
TNFR3r
5′-TexasRed ®-NH-TCC-GGA TAC TCC GTA TCC TAT TCC-
3′

Temperature cycling: denaturation at 95° C. for 5 min, then 30 amplification cycles (95° C. for 35 sec, 64° C. for 45 sec, 72° C. for 45 sec) and final incubation at 72° C. for 5 min. Two microliters of the reaction mix after the first PCR step are used as a template for the second step.

The second PCR step is carried out to produce a preferentially single-stranded product required for hybridization to oligonucleotide probes. For this, the primer TNFR1f and the fluorescently labeled primer TNFR3r are used at a ratio of 1:10. Temperature cycling was the same as at the first step, but the number of cycles was increased to 35.

Hybridization of the Amplified Labeled Product to a Biochip

A 12 μl sample from the asymmetrical PCR (˜1.2 μg ssDNA) was used for hybridization to a biochip in the following buffer: 1 M NaCl, 50 mM HEPES, pH 7.5, 5 mM Na₂EDTA. The hybridization was performed in a 30 μl hybridization chamber (Sigma) for 4 to 6 h at 37° C. The chip was washed 3 times in a buffer composed of 0.8 M NaCl, 50 mM HEPES, pH 7.0, 6 mM EDTA, 0.5% Tween 20 at 37° C. and dried.

Detection and Analysis of Hybridization Results

The detection of hybridization patterns was accomplished in an experimental device comprising a fluorescence microscope, a CCD camera, and a computer. Digital imaging was performed using the WinView software (Princeton Instruments, USA). The fluorescent signal intensities were quantitized using a customized software.

The results are analyzed as follows. Fluorescent signal intensities are compared within each column of gel spots containing oligonucleotides for one of species-specific positions of the crmB gene, either by visual inspection or using a computer software. The hybridized DNA probe is able to form a perfectly matched duplex with only one species-specific oligonucleotide within each row, forming mismatched duplexes with all the other oligos. The fluorescent signal of a perfectly matched duplex in each row is several-fold higher than the signals from mismatched duplexes. As a result, there is a hybridization pattern formed throughout the chip which is different for the 6 orthopoxvirus species, thus enabling an unambiguous interpretation of the data obtained.

The Structure of the Biochip

The biological microchip contains 15 immobilized oligonucleotides as listed in Table 1. Each of the 5 vertical columns of the biochip contains oligonucleotides for one particular species-specific position of the crmB gene. Thus, oligonucleotides for 5 species-specific positions of the gene are present on the chip, making it possible to differentiate species reliably. The resulting hybridization pattern is compared to reference patterns (FIG. 1) to find a match, according to which the result is identified.

TABLE 1

Typing oligonucleotides used for the differen-
tial diagnostics of orthopoxviruses

| | | |
|---|---|---|
| Amino acid | | |
| position | Species or | Sequence, 5′-3′, |
| No. | strain | 3′-NH₂ |

| | | | |
|---|---|---|---|
| 1 | 63, ins. | cow pox | CTA A(C/T)A CAA ACA |
| | | | CAC A-NH₂ |
| 2 | 63 | all the rest | CTA A(C/T)A CAA AAT |
| | | | GTA C-NH₂ |
| 3 | 63, subst. | rabbit pox + | CTA A(C/T)A CAC GAT |
| | | some v.v. | GTA C-NH₂ |
| 4 | 81 | all the rest | TTA CCC GCT TGT C- |
| | | | NH₂ |
| 5 | 81, subst. | monkey pox | TTA CAG GCT TGT CT- |
| | | | NH₂ |
| 6 | 81, subst. | tatera pox | TTA CCC ACT TGT CT- |
| | | | NH₂ |
| 7 | 90 | variola virus | AAG ATG CAA TAG TAA |
| | | | T-NH₂ |
| 8 | 90, subst. | camel + cow pox | AAG ATG CGA TAG TAA |
| | | | T-NH₂ |
| 9 | 90, subst. | monkey + tatera | AAG ATG TGA TAG TAA |
| | | | T-NH₂ |
| 10 | 90, del. | buff. + v.v. + | GGA AGA CGC GAT-NH₂ |
| | | rabbit | |
| 11 | 116 | variola virus | GTC TTC TTA AAG GA- |
| | | | NH₂ |
| 12 | 116, subst. | camel pox | GTA TTC TGA AAG GA- |
| | | | NH₂ |
| 13 | 116, subst. | all the rest | GTC TTC TCA AAG GA- |
| | | | NH₂ |
| 14 | 126 | all the rest | ATT TCC CAA ACA AA- |
| | | | NH₂ |
| 15 | 126, subst. | monkey pox | ATT TCT AAA ACA AA- |
| | | | NH₂ |

### EXAMPLE 1

Differential Diagnostics of Orthopoxviruses using Biochips. Theoretical Patterns and Actual Hybridization Patterns Corresponding to Them

FIG. 1 shows hybridization patterns of DNA samples from different orthopoxviruses and the respective reference patterns. It can be seen that each of the orthopoxvirus species tested is characterized by a unique hybridization pattern. Within each column, a particular spot is visually distinguished by having a maximum fluorescent signal intensity that corresponds to a perfectly matched duplex.

Advantages of the Method

The method for differential diagnostics of orthopoxviruses using a miniature biochip is advantageous by a rapid analysis time, a simple procedure for the preparation of viral DNA samples for hybridization, suitability for analysis in the field conditions as well as a relative cost-effectiveness.

### REFERENCES

1. Avakyan A. A., Altshtein A. D., Kirillova F. M., Bykovsky A. F. 1981. The ways to improve the laboratory diagnostics of smallpox. Vopr. Virusol. No. 2, 196-203.
2. Maltseva N. N. 1980. A rapid diagnostics of diseases caused by orthopox- and some herpesviruses. D.Sc. Thesis, Moscow.
3. Marennikova S. S., Yanova N. N., Zhukova O. A. 1990. Electron microscopy as a diagnostic method to monitor poxvirus infections. Zh. Microbiol. No. 8, 57-62.
4. Marennikova S. S., Nagieva F. G., Matsevich G. R., Shelukhina E. M., Khabakhpasheva N. A., Platonova G. M. 1988. Monoclonal antibodies to monkeypox virus: preparation and application. Acta Virol. 32, 19-26.
5. Lazarus A. S., Eddie A., Meyer K. F. 1937. Propagation of variola virus in the developing egg. Proc. Soc. Exp. Biol. Med. 36(1), 7-8.
6. Downie A. W., Dumbell K. R. 1947. The isolation and cultivation of variola virus on the chorioallantois of chick embryos. J. Path. Bact. 59(1-2), 189-198.
7. Marennikova S. S., Matsevich G. R., Khabakhpasheva N. A., Novokhatsky A. S., Malakhova I. V., Shelukhina E. M. 1986. Enhancing the specificity of enzyme-linked immunoassay by using a monoclonal antibody-based conjugate. Vopr. Virusol. No. 6, 689-690.
8. Noskov F. S., Marennikova S. S., Konnikova R. E. et al. 1972. The application of indirect hemagglutination reaction for the laboratory diagnostics of smallpox. Vopr. Virusol. No. 3, 347-351.
9. Meyer H., Pfeffer M. & Rziha H.-J. 1994. Sequence alterations within and downstream of the A-type inclusion protein genes allow differentiation of Orthopoxvirus species by polymerase chain reaction. J. Gen. Virol. 75, 1975-1981.
10. Roop S. L., Jin Q., Knight J. C., Massung R. F. & Esposito J. J. 1995. PCR strategy for identification and differentiation of smallpox and other orthopoxviruses. J. Clin. Microbiol. 33, 2069-2076.
11. Yershov G., Barsky V., Belgovsky A., Kirillov Eu., Kreindlin E., Ivanov L., Parinov S., Guschin D., Drobishev A., Dubiley S. & Mirzabekov A. 1996. DNA analysis and diagnostics on oligonucleotide microchips. Proc. Natl. Acad. Sci. USA 93, 4913-4918.

1. A method for the identification of virus species of the Orthopoxvirus genus by hybridizing an appropriately treated as described in step (b) orthopoxvirus DNA isolated from a test sample to a miniature biological chip, comprising the steps of
(a) providing a biochip for species diagnostics in the form of a microarray of gel spots on a glass support with subsequent immobilization of typing oligonucleotides,
(b) amplifying a fragment of the crmB gene by a two-step asymmetrical PCR using a fluorescently labeled primer to obtain single-stranded DNA for hybridization,
(c) performing DNA-DNA hybridization on said biochip, whereby a hybridization mix is incubated in a sealed reaction chamber over the microchip allowing the duplex formation between said fragment of viral DNA and the corresponding complementary oligonucleotides on the chip,
(d) performing detection and analysis of the hybridization results, whereby fluorescent signals are detected from the labeled sample using a recording device and the resulting hybridization pattern is subsequently compared to reference patterns that allow an adequate interpretation of the results.

2. The method according to claim 1, wherein said analysis of the resulting hybridization pattern is performed by comparing it to a set of standardized reference patterns specific for each species.

3. A test kit for implementing the method according to claim 1, comprising:
(a) a microchip that contains the typing oligonucleotides and is used for the diagnostics of orthopoxvirus species, and
(b) optionally, reagents for DNA isolation, primers for PCR reactions (to amplify a specific fragment of the viral crmB gene), reagents for hybridization, and a device for the detection of test results (such as a portable chip reader that employs an exciting laser beam and is equipped with a photographic camera or a CCD camera).

4. A biological microchip for implementing the method according claim 1.

5. Discriminating oligonucleotides to be immobilized on the biological microchip according to claim 4, characterized by species-specificity (complementarity) to different species of the Orthopoxvirus genus.

6. A test kit for implementing the method according to claim 2, comprising:
(a) a microchip that contains the typing oligonucleotides and is used for the diagnostics of orthopoxvirus species, and
(b) optionally, reagents for DNA isolation, primers for PCR reactions (to amplify a specific fragment of the viral crmB gene), reagents for hybridization, and a device for the detection of test results (such as a portable chip reader that employs an exciting laser beam and is equipped with a photographic camera or a CCD camera).

7. A biological microchip for implementing the method according to claim 2.
